(19) United States
(12) Patent Application Publication
(10) Pub. No.: US 2014/0052177 A1
(43) Pub. Date: Feb. 20, 2014

(54) METHOD AND APPARATUS FOR ATTACHING SOFT TISSUE TO BONE

(71) Applicant: DEPUY SYNTHES PRODUCTS, LLC, Raynham, MA (US)

(72) Inventors: Wamis Singhatat, Paoli, PA (US); Scott Larsen, Paoli, PA (US); William Miller, Paoli, PA (US); Daniel Vennard, West Chester, PA (US); Jordan P. Conley, West Chester, PA (US)

(73) Assignee: DEPUY SYNTHES PRODUCTS, LLC, Raynham, MA (US)

(21) Appl. No.: 13/837,309

(22) Filed: Mar. 15, 2013

Related U.S. Application Data
(60) Provisional application No. 61/684,075, filed on Aug. 16, 2012.

(51) Int. Cl. A61B 17/064

(52) U.S. Cl. CPC A61B 17/064
USPC 606/232

(57) ABSTRACT

A method and apparatus including at least one anchor and at least one suture assembly. The anchor comprising a body and a suture capture element formed in the body for attaching at least one suture to the anchor The suture capture element being configured so as to permit the suture to be snared by the capture element after the anchor has been attached to bone. The suture assembly including a first loop and a second loop. The suture assembly being configured to attach to soft tissue or bone with at least one of the first and second loops disposed about the suture capture element of the anchor.

### INCORPORATION BY REFERENCE

The entirety of U.S. provisional application, Ser. No. 61/684,075, filed on Aug. 16, 2012, is hereby expressly incorporated herein by reference.

### BACKGROUND

In many situations soft tissue may need to be attached (or re-attached) to bone or bone may need to be attached (or re-attached) to bone. As an example, a ligament or tendon may have been detached from bone as the result of injury, and appropriate repair may require re-attaching the ligament or tendon to its host bone. The use of sutures together with one or more suture anchors is one way of attaching soft tissue to bone. Suture anchors generally include a body that is deployed in or on bone with one or more sutures extending from the body. The sutures may then be used to secure the soft tissue to the bone, e.g., by passing the sutures through the tissue and then knotting the suture so as to hold the tissue in position relative to the bone.

Suture anchors and bone plates are commonly used to anchor soft tissue to bone. Many suture anchors and bone plates are configured such that the sutures need to be attached to the suture anchor or the bone plate either at the time of manufacture or in the operating room prior to use. In either case, the sutures are attached to the body of the suture anchor prior to deployment of the suture anchor in the body. As a result, a surgeon may be constrained by the configuration of the suture anchor once it has been deployed in the body. In other words, with many suture anchor constructions, the number and types of sutures selected by the surgeon prior to deployment in the bone significantly constrains the choices available to the surgeon after the suture anchor has been deployed in the bone. This can be a significant limitation, since in many circumstances the surgeon may wish to adjust a procedure in response to tissue conditions which may only become apparent after the procedure has commenced.

In an attempt to overcome such shortcomings, suture anchors and bone plates have been suggested that permit sutures to be loaded into the suture anchor and bone plate after deployment in the bone. Such suture anchors and bone plates are disclosed in U.S. Publication No. 2012/0150235 by Snyder et al. One of the suture anchors disclosed in the '235 application includes a body having a proximal end, a distal end, an outer surface, and an axial recess extending from the proximal end toward the distal end. A suture capture element in the form of a flexible finger or crossbar extends from the body and across the axial recess such that when a distally directed force is applied to the crossbar, a portion of a suture may be passed by the crossbar and when a proximally directed force is applied to the suture, the crossbar is captured to limit proximal motion of the crossbar and thereby capture the suture. Similarly, a bone plate disclosed in the '235 application includes a similar type of suture capture element.

Knotless suture assemblies have been suggested that permit anatomical structures to be connected with one another without requiring the tying of knots. Such suture assemblies are disclosed in U.S. Publication No. 2012/0150233 by Manos et al.

### BRIEF DESCRIPTION OF THE DRAWINGS

To assist those of ordinary skill in the relevant art in making and using the inventive concepts disclosed herein, reference is made to the appended drawings and schematics, which are not intended to be drawn to scale, and in which like reference numerals may refer to the same or similar elements for consistency. For purposes of clarity, not every component may be labeled in every drawing. Certain features and certain views of the figures may be shown exaggerated in scale or in schematic in the interest of clarity and conciseness. In the drawings:

FIG. 1 is a schematic illustration of a suture anchor assembly constructed in accordance with the inventive concepts disclosed herein.

FIG. 2A is a perspective view of a suture anchor insert shown positioned in a bone plate.

FIG. 2B is a cross sectional view of the suture anchor insert of FIG. 2B.

FIG. 3 is top plan view of another embodiment of a suture anchor assembly constructed in accordance with the inventive concepts disclosed herein.

FIG. 4 is a front elevational view of an exemplary embodiment of a suture anchor.

FIG. 5 is a side elevational view of the suture anchor of FIG. 4.

FIG. 6 is a top plan view of the suture anchor of FIG. 4.

FIG. 7 is a perspective view of an exemplary embodiment of an anchor driver.

FIG. 8 is an elevational view of the anchor driver.

FIG. 9 is a front elevational view of an exemplary embodiment of a suture threader.

FIG. 10 is a front elevational view of the suture threaded of FIG. 9 illustrated positioned in a suture anchor.

FIG. 11 is a side elevational view of the suture threaded of FIG. 9 illustrated positioned in the suture anchor.

FIG. 12 is a front elevational view of the suture threaded of FIG. 9 illustrated with the crossbar of the suture anchor positioned at a lower end of the suture threader

FIG. 13 is an elevational view of the suture anchor of FIG. 4 shown with a suture-binding plug inserted therein.

FIG. 14 is an elevational view of the suture anchor of FIG. 4 shown with another embodiment of a suture-binding plug inserted therein

FIG. 15 is a perspective view of another exemplary embodiment of a suture anchor.

FIG. 16 is a top plan view of the suture anchor of FIG. 15.

FIG. 17 is a side elevational view of another embodiment of suture threaded.

FIG. 18 is front elevational view of the suture threader of FIG. 17

FIG. 18A is front elevational view of another embodiment of a suture threader.

FIG. 19 is an elevational view illustrating the suture threaded of FIG. 17 positioned in the suture anchor of FIG. 15.

FIG. 20 is a perspective view illustrating use of the suture threader of FIG. 17 in the suture anchor of FIG. 15.

FIG. 21 is a top plan view showing the suture threader of FIG. 17 in the suture anchor of FIG. 15.

FIG. 22 is a cross sectional view another embodiment of an exemplary embodiment of a suture anchor.

FIG. 23 is a perspective view of another embodiment of a bone plate.

FIG. 24 is a top plan view of the bone plate of FIG. 23.

FIG. 25 is a side elevational view of the bone plate of FIG. 23.

FIG. 26 is cross sectional view of a portion of the bone plate of FIG. 23.

FIG. 27 is a top plan view of a portion of another embodiment of a bone plate.

FIG. 28 is a side elevational view of the bone plate of FIG. 27.

FIG. 29 is a cross sectional view of the bone plate of FIG. 27 prior to a suture being connected thereto.

FIG. 30 is a cross sectional view of the bone plate of FIG. 27 shown with the suture connected thereto.

FIG. 31 is an elevational view of a plug.

FIG. 32 is an elevational view of the plug of FIG. 31 shown positioned in a suture anchor in a collapsed condition.

FIG. 33 is an elevational view of the plug of FIG. 32 shown in an expanded condition.

FIG. 34 is an elevational view of an exemplary embodiment of a suture assembly constructed in accordance with the inventive concepts disclosed herein.

FIG. 34A is an enlarged, perspective view of a portion of the suture assembly of FIG. 34.

FIG. 35 is an elevational view of another exemplary embodiment of a suture assembly.

FIG. 36 is an elevational view of another exemplary embodiment of a suture assembly.

FIG. 37 is an elevational view of yet another exemplary embodiment of a suture assembly.

FIG. 38 is an elevational view of an exemplary embodiment of a suture assembly shown connected to soft tissue.

FIG. 39A is an elevational view of another embodiment of a suture assembly shown in a collapsed condition and connected to a suture anchor.

FIG. 39B is an elevational view of the suture assembly of FIG. 39A shown in an expanded condition.

FIGS. 40-42 are schematic illustrations of several embodiments of suture passers.

FIGS. 43A-43F are schematic diagrams illustrating steps of attaching a soft tissue with the suture anchor of FIGS. 1-3 with the suture assembly of FIG. 34.

### DETAILED DESCRIPTION OF EXEMPLARY EMBODIMENTS

Before explaining at least one embodiment of the presently disclosed and claimed inventive concepts in detail, it is to be understood that the presently disclosed and claimed inventive concepts are not limited in their application to the details of construction, experiments, exemplary data, and/or the arrangement of the components set forth in the following description or illustrated in the drawings. The presently disclosed and claimed inventive concepts are capable of other embodiments or of being practiced or carried out in various ways. Also, it is to be understood that the phraseology and terminology employed herein is for purpose of description and should not be regarded as limiting.

In the following detailed description of embodiments of the inventive concepts, numerous specific details are set forth in order to provide a more thorough understanding of the inventive concepts. However, it will be apparent to one of ordinary skill in the art that the inventive concepts within the disclosure may be practiced without these specific details. In other instances, certain well-known features may not be described in detail to avoid unnecessarily complicating the instant disclosure.

As used herein, the terms “comprises,” “comprising,” “includes,” “including,” “has,” “having,” or any other variation thereof, are intended to cover a non-exclusive inclusion. For example, a process, method, article, or apparatus that comprises a list of elements is not necessarily limited to only those elements but may include other elements not expressly listed or inherently present therein.

Unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

The term “and combinations thereof” as used herein refers to all permutations or combinations of the listed items preceding the term. For example, “A, B, C, and combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. A person of ordinary skill in the art will understand that typically there is no limit on the number of items or terms in any combination, unless otherwise apparent from the context.

In addition, use of the “a” or “an” are employed to describe elements and components of the embodiments herein. This is done merely for convenience and to give a general sense of the inventive concepts. This description should be read to include one or at least one and the singular also includes the plural unless it is obvious that it is meant otherwise.

The use of the terms “at least one” and “one or more” will be understood to include one as well as any quantity more than one, including but not limited to each of, 2, 3, 4, 5, 10, 15, 20, 30, 40, 50, 100, and all integers and fractions, if applicable, therebetween. The terms “at least one” and “one or more” may extend up to 100 or 1000 or more, depending on the term to which it is attached; in addition, the quantities of 100/1000 are not to be considered limiting, as higher limits may also produce satisfactory results.

Further, as used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein qualifiers such as “about,” “approximately,” and “substantially” are intended to signify that the item being qualified is not limited to the exact value specified, but includes some slight variations or deviations therefrom, caused by measuring error, manufacturing tolerances, stress exerted on various parts, wear and tear, and combinations thereof, for example.

As used herein, the term “patient” is meant to include all organisms, whether alive or dead, including any species having soft tissues and bones. For example, a method according to the inventive concepts disclosed herein may be used to repair a soft tissue detachment injury in a living human, horse, cow, sheep, cat, dog, and the like. In another example, a method according to the inventive concepts disclosed herein may be used in a non-living organism to train medical personnel in surgical techniques. As yet another example, a method according to the instant disclosure may be used to implant medical devices such as replacement joints, pacemakers, and the like, into an organism by anchoring such devices to a bone. As yet another example, a method according to the inventive concepts disclosed herein may be used to repair rotator cuff instabilities and tears in shoulder surgery, or to repair various knee, elbow, hip, wrist, ankle, or other soft tissue detachment and joint injuries.

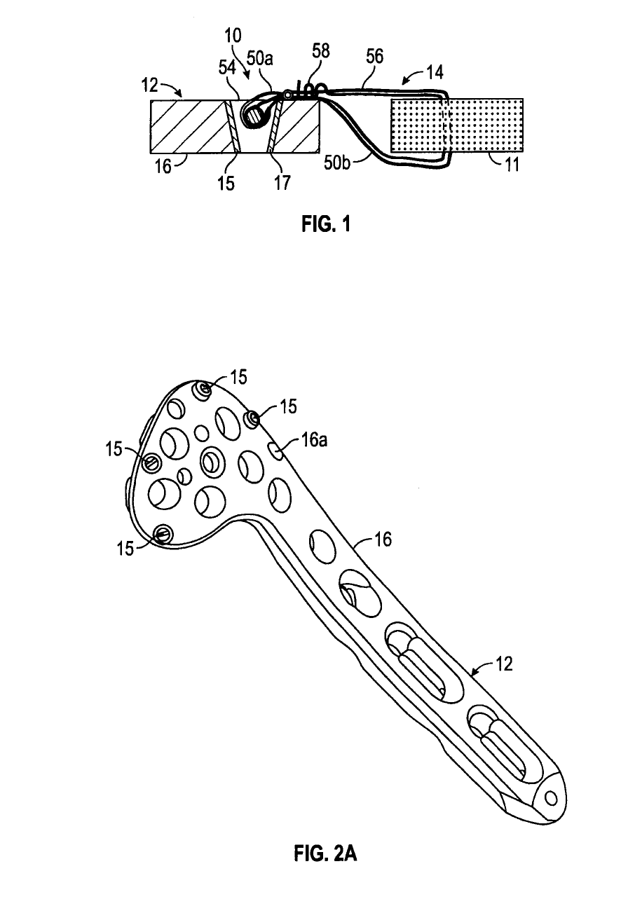

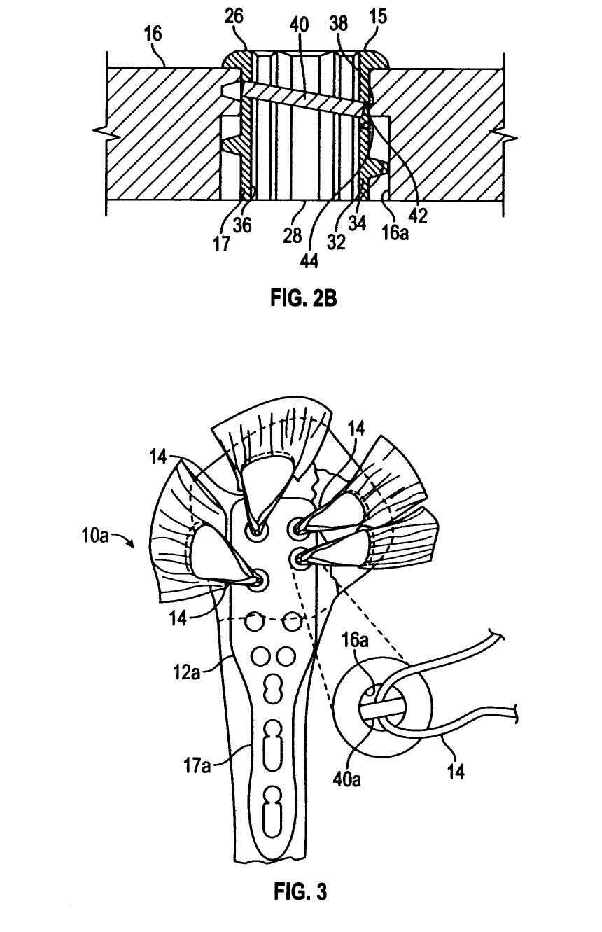

Referring to the drawings, and more particularly to FIGS. 1 and 2A, a suture anchor assembly 10 constructed in accordance with the inventive concepts disclosed herein is shown connected to a segment of soft tissue 11 (e.g., a tendon, a ligament, cartilage, the annulus of an intervertebral disc, or the like).

The suture anchor assembly 10 includes an anchor 12 and a suture assembly 14. With reference to FIG. 2B, the suture anchor assembly 10 broadly includes an anchor insert 15 positioned in a bone plate 16. The anchor insert 15 has a body 17 configured to be slidably inserted into one of the holes 16a of the bone plate 16. The body 17 has a proximal end 26, a distal end 28, and a sidewall 32 extending between the proximal end 26 and the distal end 28. The sidewall 32 is provided with at least one plate engaging member 34. The sidewall 32 defines a recess 36 within the body 15 of the suture anchor insert 10. The sidewall 32 is further provided with a transverse edge 38. Opposite the transverse edge 38, the sidewall 32 is provided with a suture capture element in the form of a crossbar 40.

The crossbar 40 extends inwardly therefrom across the recess 36 such that a free end 42 of the crossbar 40 is normally disposed distally of the transverse edge 38. The crossbar 40 is flexible such that when a distally directed force is applied to the crossbar 40, the free end 42 of the crossbar 40 is positioned in the recess 36 and when a proximally directed force is applied to the crossbar 40 such as by a suture which is looped over the crossbar 40, the free end 42 of the crossbar 40 engages with the transverse edge 38 to limit proximal motion of the crossbar 40.

The sidewall 32 of the body 14 may be provided with a crossbar window 44 which is in communication with the recess 36. The crossbar window 44 has a proximal side and a distal side where the proximal side of the crossbar window 44 defines the transverse edge 38. The crossbar 40 extends across the recess 36 such that the free end 42 of the crossbar 40 is normally disposed in the crossbar window 44.

The suture assembly 14 includes a pair of loops 50a and 50b formed on opposing ends of the suture assembly 14. The loops 50a and 50b are adapted to be captured by the suture capture element of the anchor either before or after the anchor has been deployed in bone. The suture assembly 14 further includes a region whereby a first strand segment 54 is woven into a second strand segment 56 to define a stitch lock 58 which permits the tension of the suture assembly 14 to be adjusted without requiring the tying of a knot.

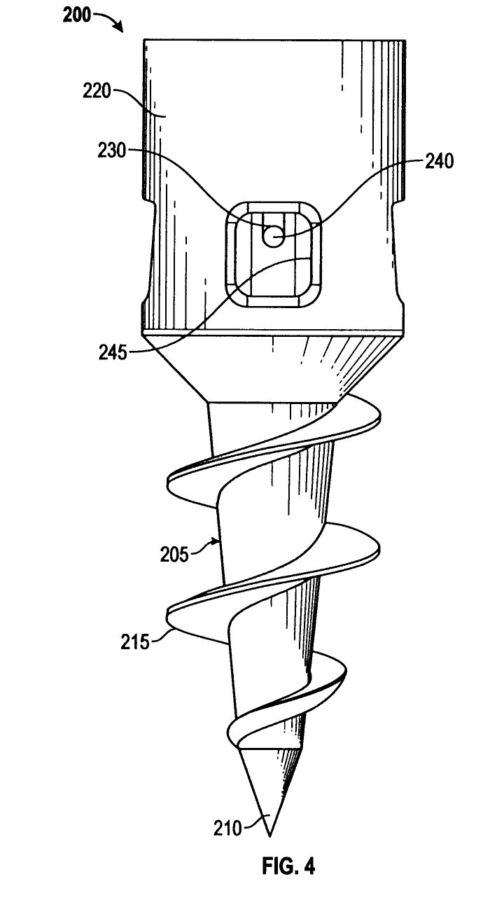

FIG. 3 illustrates another embodiment of a suture anchor assembly 10a constructed in accordance with the inventive concepts disclosed herein. The suture anchor assembly 10 includes an anchor 12a and a suture assembly 14. The anchor 12a broadly includes a body 17a in the form a bone plate having a plurality of holes 16a extending therethrough. A suture capture element in the form of a flexible finger or crossbar 40a extends from the body and across the holes 16a such that when a distally directed force is applied to the crossbar 40a, a portion of a suture may be passed by the crossbar 40a and when a proximally directed force is applied to the suture assembly 14, the crossbar 40a is captured to limit proximal motion of the crossbar 40a and thereby capture the suture assembly 14.

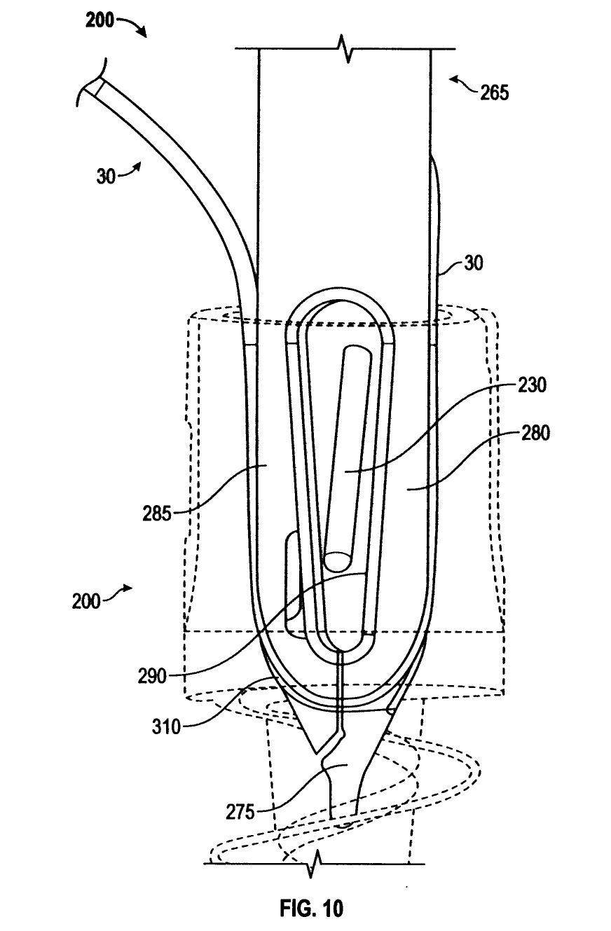

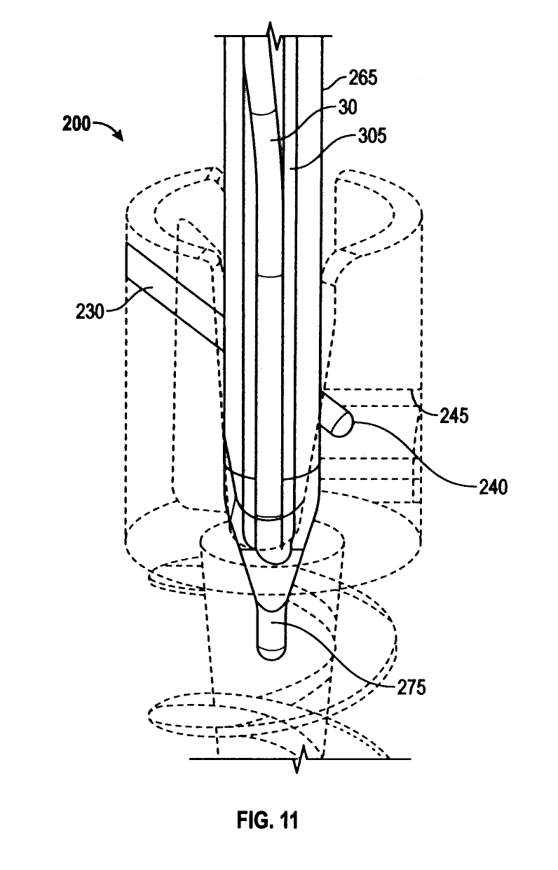

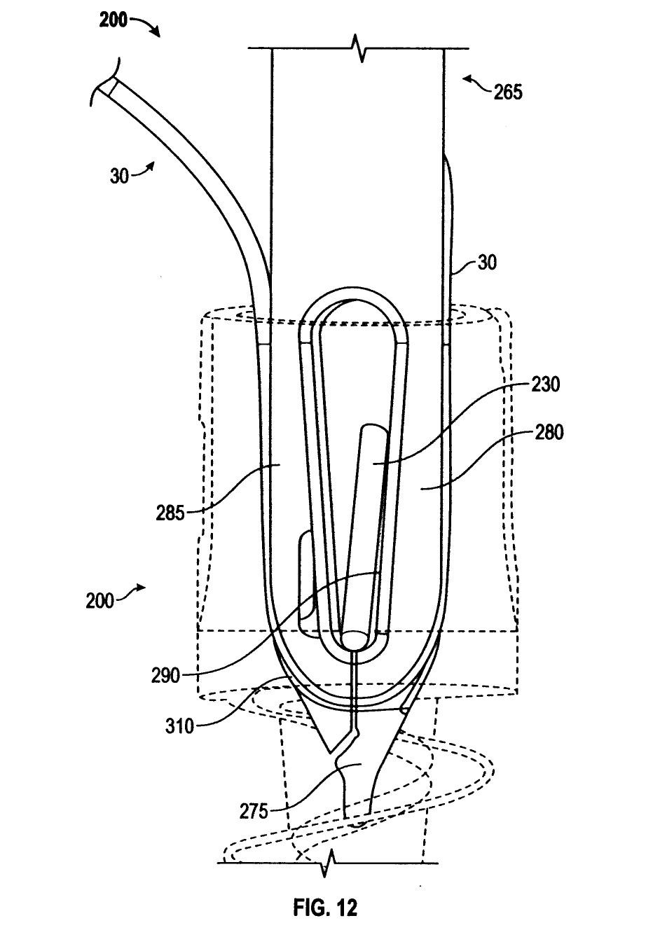

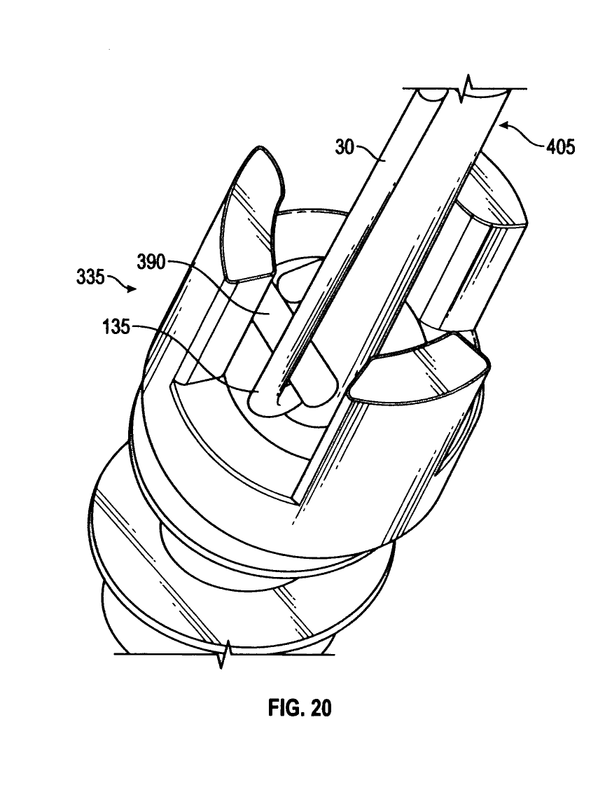

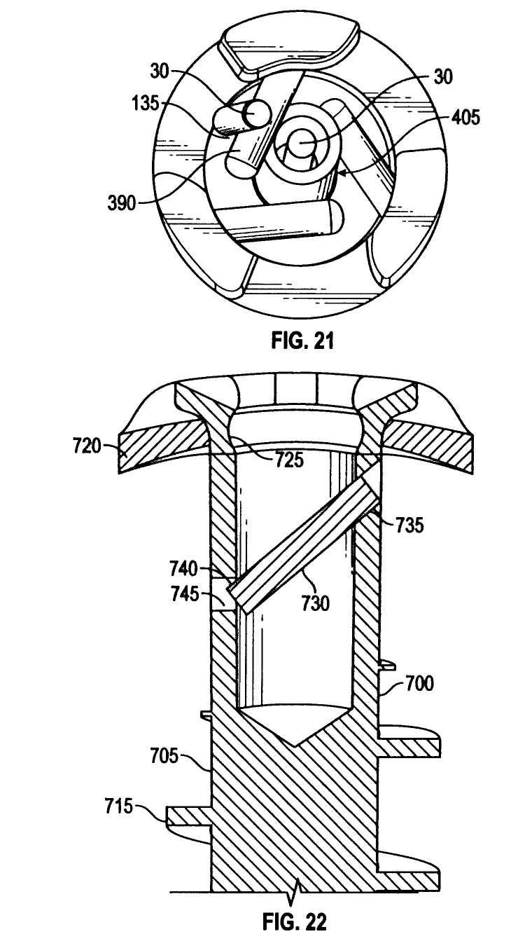

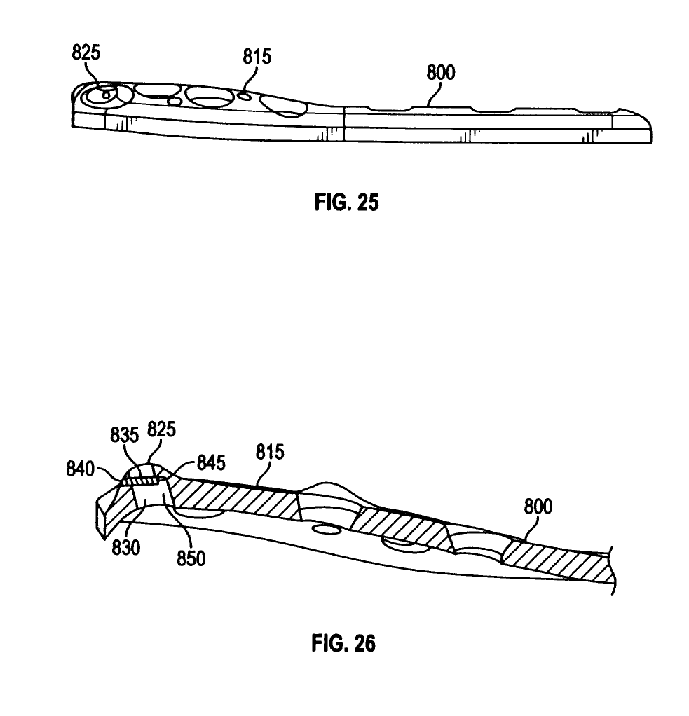

FIGS. 1-3 generally illustrate examples of suture anchors that may be employed with the suture anchor assemblies disclosed herein. Other examples of suture anchors which employ suture capture elements are illustrated in FIGS. 4-34 and disclosed in U.S. Publication. Nos. 2009/0088798 and 2012/0150235, as well as U.S. patent applications Ser. Nos. 13/430,201 and 61/683,382, each of which is hereby incorporated herein by reference.

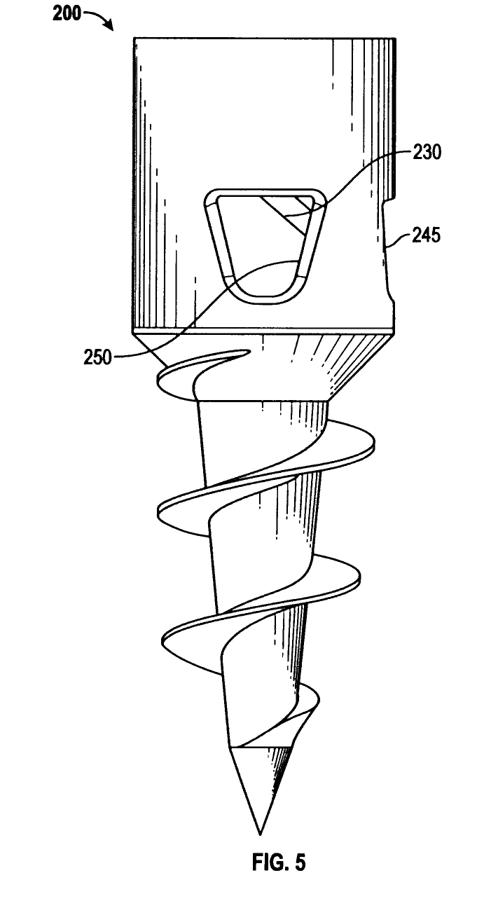

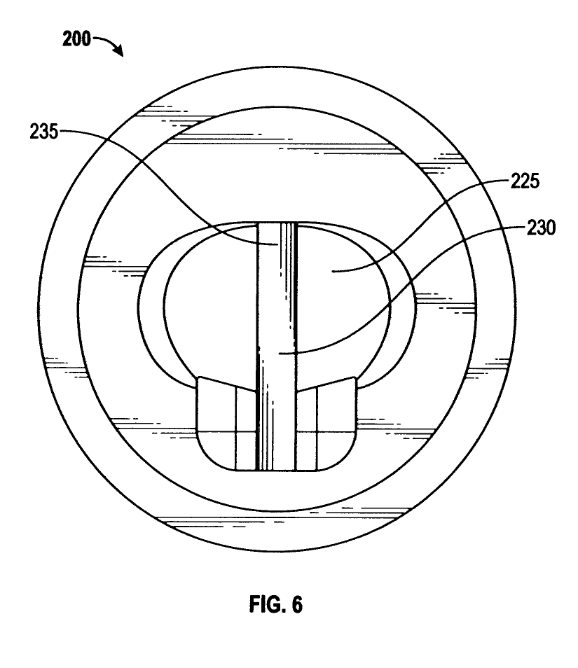

With reference to FIGS. 4-6, an anchor 200 formed in accordance with the present invention is shown therein. Anchor 200 generally comprises a shaft 205 terminating in a pointed tip 210 and having screw threads 215 thereon. Anchor 200 also comprises a head 220 having an axial recess 225 formed therein. Axial recess 225 has a non-circular (e.g., ovoid) cross-section. A flexible crossbar 230 extends across axial recess 225. More particularly, a flexible crossbar 230 comprises a fixed end 235 which is secured to head 220 and a free end 240, whereby to form a cantilever construction. Flexible crossbar 230 extends at a transverse angle to the longitudinal axis of anchor 200. More particularly, flexible crossbar 230 descends distally as it extends across axial recess 225, in the manner shown in FIGS. 5 and 6, so that flexible crossbar 230 has its free end 240 disposed distally of its opposing fixed end 235. Head 220 of anchor 200 also comprises a crossbar window 245. The free end 240 of flexible crossbar 230 extends into crossbar window 245 for reasons which will hereinafter be discussed. Head 220 also comprises a pair of diametrically-opposed side windows 250.

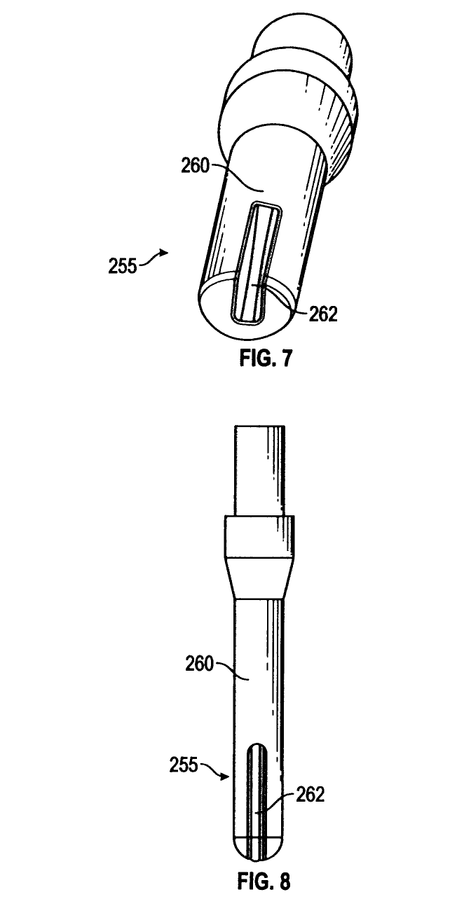

Anchor 200 is intended to be screwed into bone by a driver 255 (FIGS. 7 and 8). More particularly, driver 255 generally comprises a shaft 260 having a non-circular (e.g., ovoid) cross-section. The cross section of shaft 260 is coordinated with the cross-section of axial recess 225 in anchor 200 in order that driver 255 can be used to turn anchor 200, whereby to screw anchor 200 into bone. Driver 255 includes a slot 262 for accommodating flexible crossbar 230 of anchor 200, as will hereinafter be discussed in further detail.

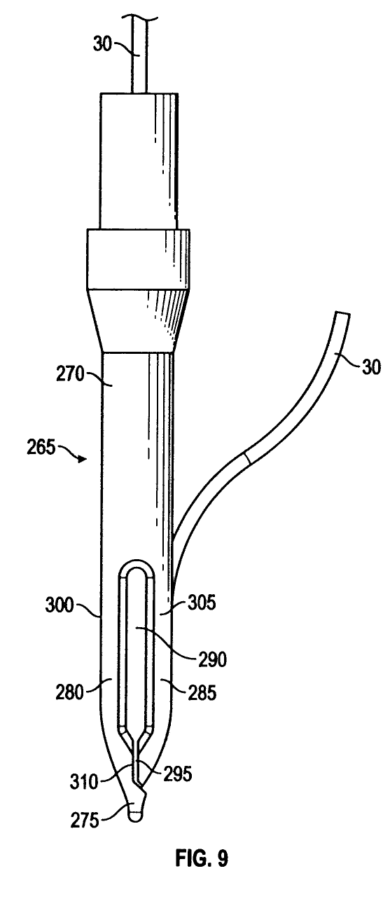

Referring next to FIGS. 9-12, there is shown a suture threader 265 which may be used in conjunction with anchor 200. Suture threader 265 generally comprises a shaft 270 terminating in a distal tip 275. Shaft 270 is cut along its distal end so as to produce a pair of parallel fingers 280, 285 which are separated intermediate their length by a window 290. Preferably fingers 280, 285 re-converge distal to window 290 at an interface 295. A surface groove 300 is formed in finger 280 for receiving suture 30. Another surface groove 305 is formed in finger 285 for receiving another portion of suture 30. An opening 310 in finger 280 permits suture 30 to pass from surface groove 300 to surface groove 305. It will be appreciated that suture 30 will be configured in the form of a distal loop in the region where suture 30 passes from surface groove 300, through opening 310 and into surface groove 305, as will hereinafter be discussed below. At least finger 285, and preferably finger 280 as well, is formed out of a resilient material, such that finger 280 can spring toward and away from finger 285.

Anchor 200, driver 255 and suture threader 265 may be used as follows. First, a pilot hole is preferably made in the bone which is to receive anchor 200, although in some circumstances the pilot hole may be omitted. Then driver 255 is used to screw anchor 200 into the bone. This is done by advancing the distal end of driver 255 into axial recess 225 of anchor 200, with flexible crossbar 230 received in slot 262 in driver 255, and then turning driver 255 so as to screw anchor 200 into the bone.

Next, suture threader 200, with suture 30 disposed thereon (i.e., seated within surface groove 300, opening 310 and surface groove 305), is advanced into axial recess 225 of anchor 200. As this occurs, and looking now at FIGS. 10-12, the distal end of suture threader 265 engages flexible crossbar 230 and, by virtue of this engagement, causes the free end 240 of flexible crossbar 230 to flex downwardly, “skidding” along the exterior surface of suture threader 265, until the free end 240 of flexible crossbar 230 “pops” through window 290. This action provides tactile feedback to the surgeon, confirming that flexible crossbar 230 is seated in window 290. Thereafter, suture threader 265 is withdrawn proximally from axial recess 225 of anchor 200. As this occurs, fingers 280 and 285 of suture threader 265 engage flexible crossbar 230, causing at least finger 285 to flex outward so as to permit flexible crossbar 230 to pass by the bifurcated fingers 280, 285. However, as this occurs, flexible crossbar 230 catches suture loop 135 formed at the distal end of suture 30, causing suture 30 to be captured on anchor 200. In this respect it should be appreciated that by configuring the anchor 200 so that free end 240 of flexible crossbar 230 is normally disposed within crossbar window 245, any proximal motion of flexible crossbar 230 during retraction of suture threader 265 is limited by its engagement with the proximal surface of crossbar window 245, thereby ensuring that suture 30 remains engaged on flexible crossbar 230. Alternatively, if desired, crossbar window 245 can be replaced by another structure providing a transverse edge to limit proximal motion of flexible crossbar 230, e.g., a transverse bar.

Side windows 250 formed in anchor 200 permit fluids to pass from the interior of the bone through the anchor so as to reach the soft tissue being re-attached to the bone.

The foregoing procedure may thereafter be repeated as desired so as to attach additional lengths of suture 30 to the deployed anchor 200.

Thus it will be seen that anchor 200, driver 255 and suture threader 265 permit anchor 200 to be deployed in a bone and a suture 30 to be thereafter attached to that anchor 200, so that soft tissue may be attached to the bone using the anchor 200 and suture 30.

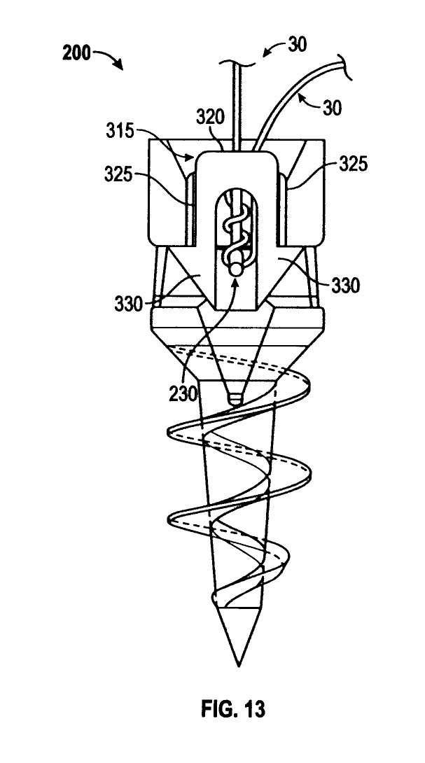

If desired, and looking next at FIG. 13, a suture-binding plug may be inserted into the proximal end of anchor 200 so as to fix suture 30 relative to the anchor 200. More particularly, in this embodiment of the inventive concepts disclosed herein, the plug may take the form of a cap 315 comprising a bridge portion 320 and a pair of descending legs 325 each terminating in a barb 330. Barbs 330 are intended to seat in side windows 250 of anchor 200, whereby to lock cap 315 to anchor 200, fixing suture 30 to anchor 200 in the process.

If desired, cap 315 can include a longitudinal bore for passing suture 30 therethrough. With this construction, cap 315 can be loaded onto the free ends of suture 30 and then slid down the suture 30 and into position on the anchor 200. By interfacing cap 315 with the suture 30 in this manner, cap 315 can be quickly and easily directed into its proper position without the risk of becoming a loose element within the body.

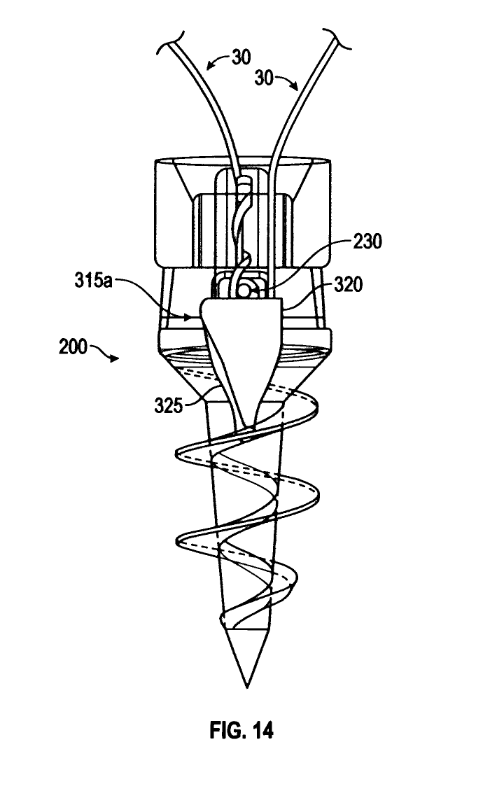

Looking next at FIG. 14, the suture-binding plug may also take the form of a cap 315a which omits barbs 330, in which case legs 325 compress into a lower portion of axial recess 225, with flexible crossbar 230 securing bridge portion 320 to the anchor 200, and with bridge portion 320 securing suture 30 to the anchor 200.

FIGS. 15 and 16 show still another anchor 335 formed in accordance with the inventive concepts disclosed herein. Anchor 335 generally comprises a shaft 360 terminating in a pointed tip 365 and having screw threads 370 thereon. Anchor 335 also comprises a head 375, at least part of which is formed by a plurality of upstanding spaced elements 380, and having an axial recess 385 formed therein. Axial recess 385 can have a non-circular (e.g., ovoid) cross-section so that it can be turned with a driver having a shaft with a non-circular (e.g., ovoid) cross-section, whereby to turn anchor 335 into bone. Alternatively, anchor 335 can be turned by a driver having a distal end having a counterpart disposition to the plurality of upstanding spaced elements 380, whereby the driver can turn the anchor 335. A plurality of flexible crossbars 390 extend across axial recess 385. Flexible crossbars 390 each comprise a fixed end 395 secured to anchor 335 and a free end 400, whereby to form a cantilever construction. Flexible crossbars 390 extend at a transverse angle to the longitudinal axis of anchor 335. More particularly, flexible crossbars 390 descend distally as they extend across axial recess 385, in the manner shown in FIG. 15, so that flexible crossbars 390 have their free ends 400 disposed distally of their opposing fixed ends 395.

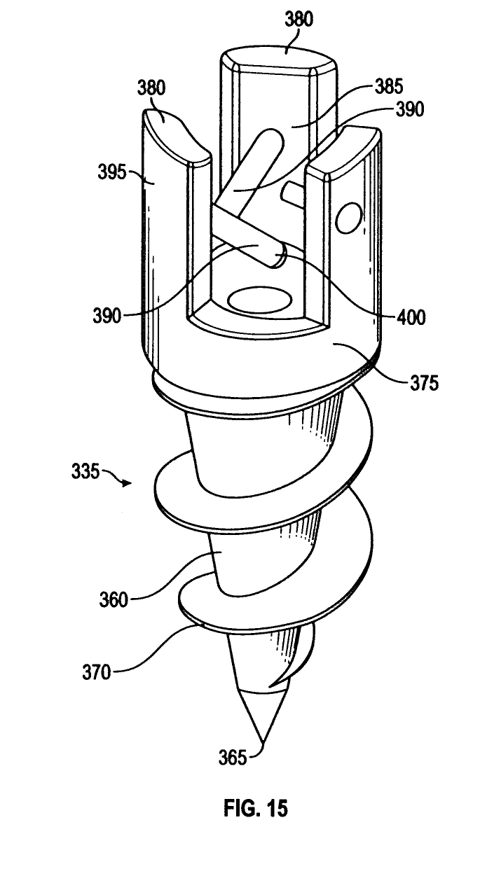

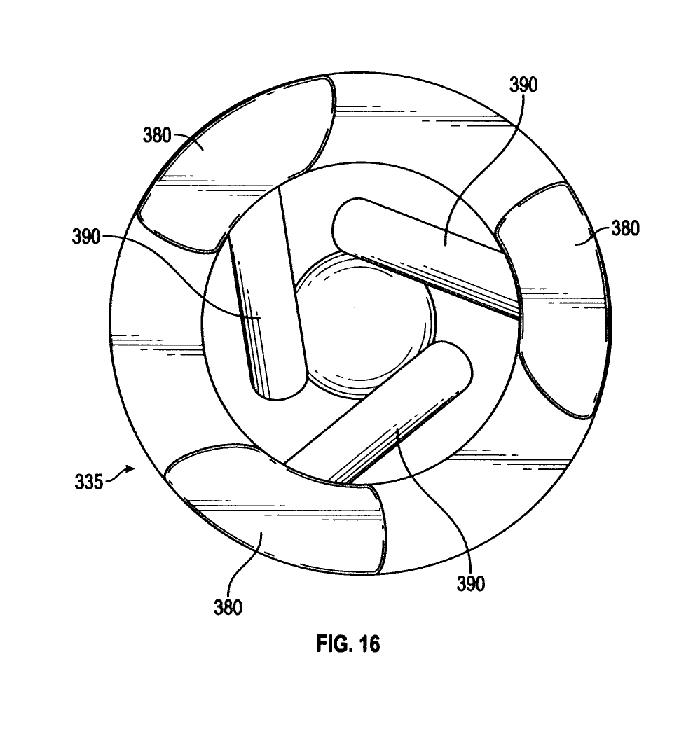

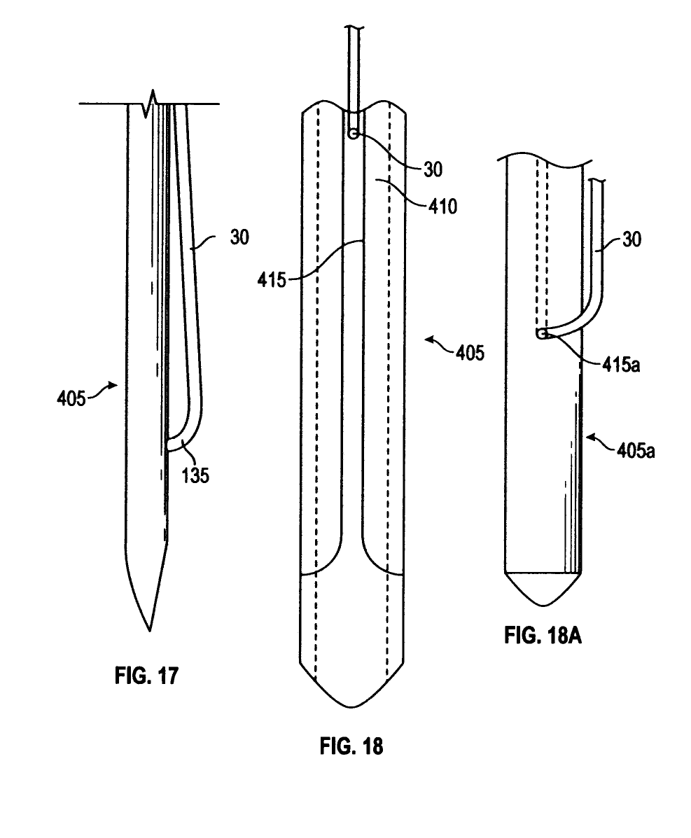

Referring now to FIGS. 17 and 18, there is shown a suture threader 405 which may be used to attach a suture 30 to anchor 335. Suture threader 405 comprises a hollow cannula having an interior lumen 410 and a slot 415 formed in its distal end. A suture 30 may be threaded down interior lumen 410, out slot 415 and then back alongside the exterior of the suture threader, in the manner shown in FIGS. 17 and 18. As a result of the natural resiliency of suture 30, a portion of a distal loop 135 of suture 30 will stand laterally displaced from the hollow cannula, such that it can be caught by one of the flexible crossbars 390.

FIG. 18A shows a suture threader 405a generally similar to suture threader 405 shown in FIGS. 17 and 18, except that slot 415 is replaced by a hole 415a.

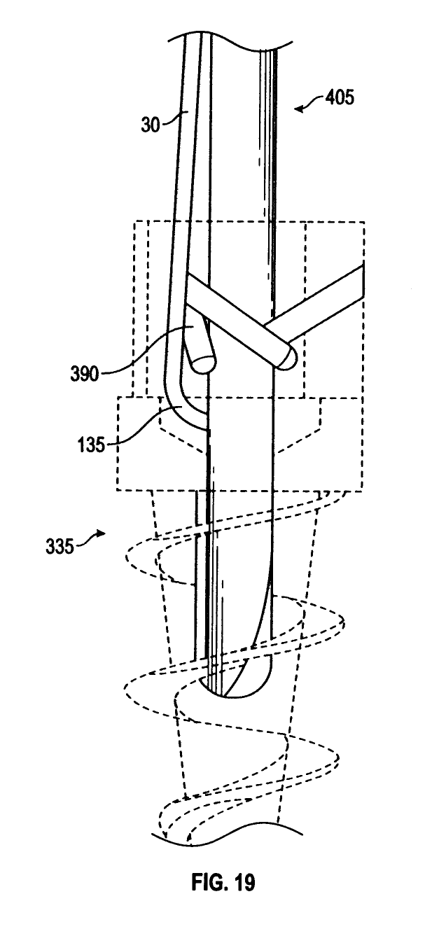

In use, and in reference to FIGS. 19-21, anchor 335 is screwed into the bone, and then a suture 30 is attached to the anchor 335 using suture threader 405. This is done by advancing the distal end of suture threader 405 into axial recess 385 of anchor 335 until suture loop 135 slips over free end 400 of a flexible crossbar 390. Then suture threader 405 is retracted, leaving suture 30 attached to anchor 335. Thus, anchor 335 and suture threader 405 permit anchor 335 to be deployed in bone and a suture 30 to be thereafter attached to that anchor 335, so that soft tissue may be attached to the bone using the anchor 335 and suture 30.

As discussed above, the screw threads of the various anchors disclosed above serve to secure the anchor to the bone. However, it should also be appreciated that other mechanisms may be used to secure the body of the anchor to the bone. Thus, by way of example but not limitation, barbs, ribs, teeth and/or other anchor-securing mechanisms of the sort well known in the art may be incorporated on the body of the suture anchor so as to ensure that the suture anchor remains secured in the bone. In addition to the foregoing, other approaches can be used to secure the body of the anchor in the bone, e.g., the body can be hammered into the bone like a nail, or the anchor can be toggled upon entry into the bone so as to prevent its withdrawal.

FIG. 22 shows an embodiment of a bone screw 700 which may be employed to secure repair bones and to secure bone plates, surgical implants, and prosthesis. The bone screw 700 generally comprises a shaft 705 terminating in a pointed tip 710 and having screw threads 715 thereon. Bone screw 700 also comprises a head 720 having an axial recess 725 formed therein. Axial recess 725 has a non-circular (e.g., ovoid) cross-section. A flexible crossbar 730 extends across axial recess 725. More particularly, a flexible crossbar 730 comprises a fixed end 735 which is secured to shaft 705 and a free end 740, whereby to form a cantilever construction. Flexible crossbar 730 extends at a transverse angle to the longitudinal axis of bone screw 700. More particularly, flexible crossbar 730 descends distally as it extends across axial recess 725, in the manner shown in FIG. 22 so that flexible crossbar 730 has its free end 740 disposed distally of its opposing fixed end 735. The head or shaft 705 of the bone screw 700 also comprises a crossbar window 745. The free end 740 of flexible crossbar 730 extends into crossbar window 745.

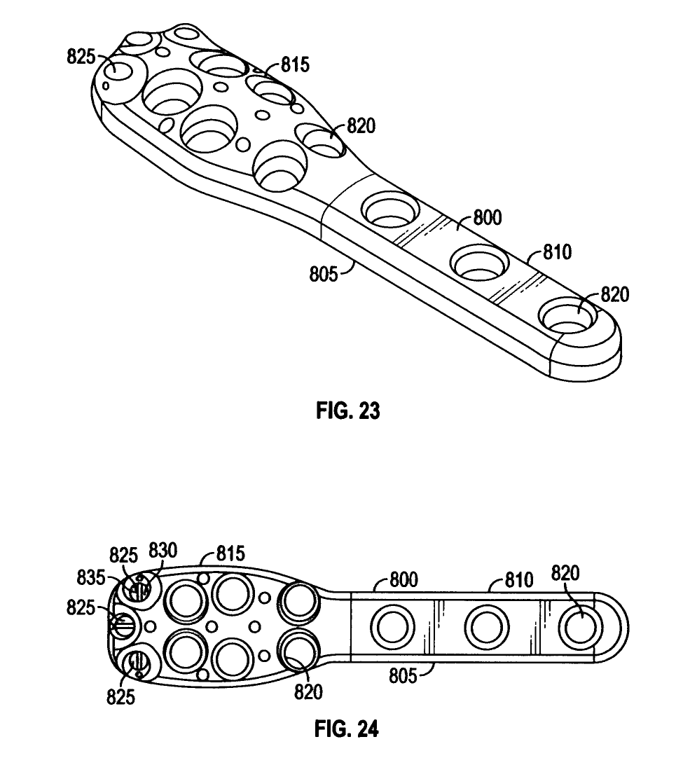

Referring now to FIGS. 23-26, a bone plate 800 constructed in accordance with the inventive concepts disclosed herein is illustrated. The bone plate 800 may be used for fixation of the long bones, such as, for example, fixation of the proximal humerus. The bone plate 800 may include a body 805 with an elongated shaft 810 with a length greater than its width and a head 815, preferably spoon-shaped, with a width which is greater than the width of the elongated shaft 810. The elongated shaft 810 and the head 815 may have a common longitudinal axis and may be interconnected by a transition area. The transition area may widen from the width of the elongated shaft 810 to the width of the head 815

A plurality of screw holes 820 may be located in the elongated shaft 810 and in the head 815. The screw holes 820 provided in the elongated shaft 810 and the head 815 may be threaded on non-threaded.

The head 815 is shown to include a plurality of suture capture elements 825. The suture capture elements 825 include a window 830 formed in the head 815. A flexible crossbar 835 extends across the window 830. More particularly, a flexible crossbar 835 comprises a fixed end 840 which is secured to the head 815 and a free end 845, whereby to form a cantilever construction. Flexible crossbar 835 extends at a transverse angle to the longitudinal axis of the body 805 of the bone plate 800. More particularly, flexible crossbar 835 descends distally as it extends across the window 830, in the manner shown in FIG. 26 so that flexible crossbar 835 has its free end 845 disposed distally of its opposing fixed end 840. The body 805 further may have a recess 850 in which the free end 845 of the flexible cross bar 835 is disposed.

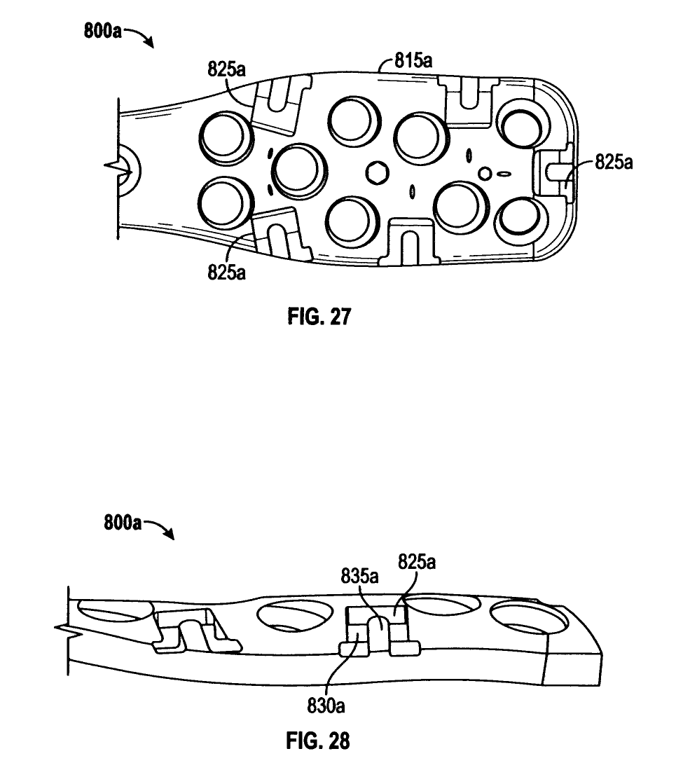

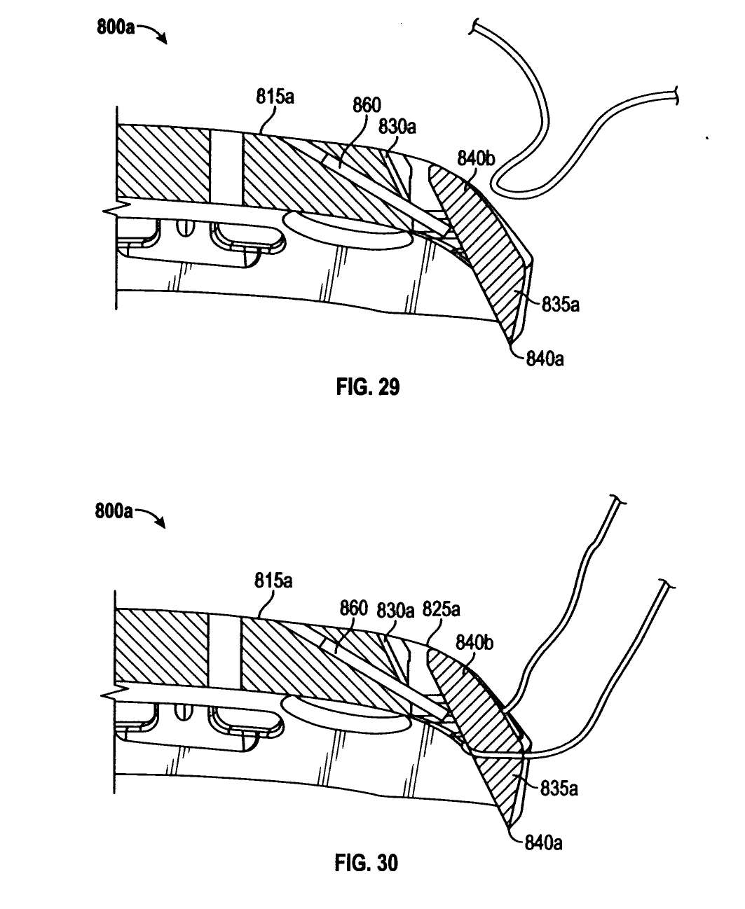

FIGS. 27-30 illustrate another version of a bone plate 800a constructed in accordance with the inventive concepts disclosed herein. The bone plate 800a is similar in construction to the bone plate 800 except as noted below. In particular, the bone plate 800a includes a plurality of suture capture elements 825a. The suture capture elements 825a include a window 830a formed in the head 815a. A rigid protrusion or peninsula 835a extends inwardly into the window 830a from a position proximate an edge of the head 815a. More particularly, the protrusion 835a comprises a fixed end 840a which is secured to the head 815a and a free end 840b. The protrusion 835a extends at an angle to the longitudinal axis of plate 800a. More particularly, protrusion 835a angles upwardly as it extends from the edge of the head 830a and into the window 830a. Moreover, the suture capture elements 825a may further include a flexible crossbar 860 (FIG. 29), which may be implemented and function similar to the crossbar 40, for example.

It should be understood that proximal humerus bone plates are used as a non-limiting example of how this technology may be employed. It should be understood that this technology may be employed many places in which surgical implants are asked to engage surgical suture.

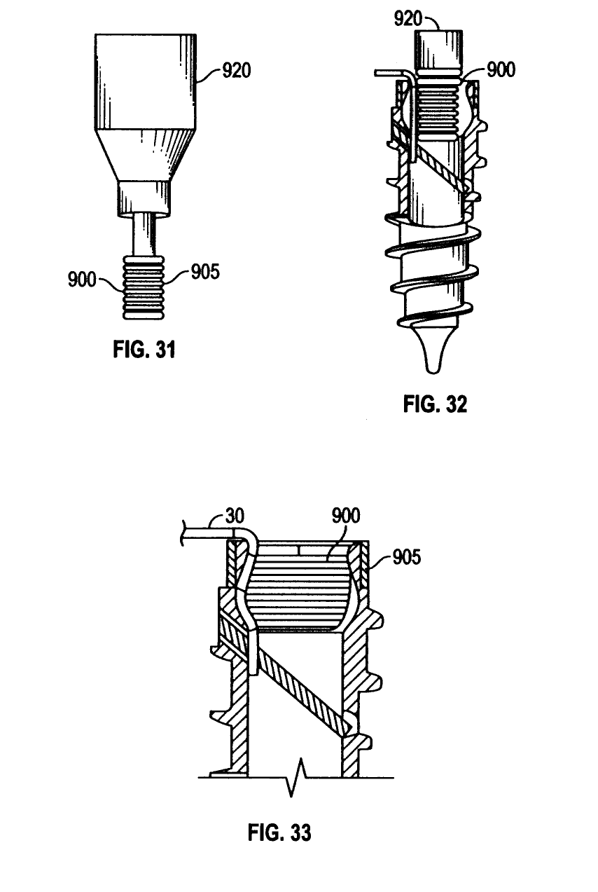

Referring to FIGS. 31-32, a suture may be secured in any hardware enhanced with the current technology using a plug as described above. Another embodiment of a plug 900 is illustrated in FIG. 33. In this embodiment, a ribbed collar 905 is expanded within the lumen of the hardware thereby applying pressure to the suture 30 and securing it in place.

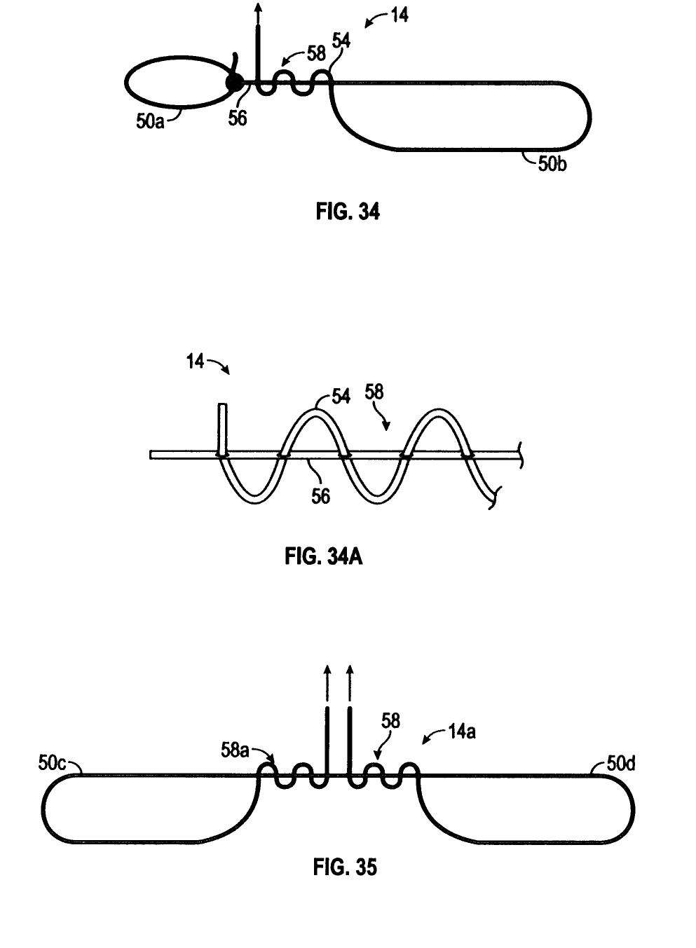

Referring now to FIGS. 34 and 34A, the suture assembly 14 is illustrated in more detail. The suture assembly 14 includes a pair of loops 50a and 50b formed on opposing ends of the suture assembly 14. The loops 50a and 50b are adapted to be captured by the suture capture element of the anchor, either before or after the anchor has been deployed in bone, and before or after the suture assembly 14 has been threaded through the soft tissue or another anchor assembly. The suture assembly 14 further includes a region whereby a first segment of a strand of suture 54 is threaded or woven into a second segment of a strand of suture 56 to define a stitch lock 58 which permits the tension of the suture assembly 14 to be adjusted without requiring the tying of a knot. When the second strand segment 56 is in tension at a level that is at least equal to a threshold level of tension, the suture assembly 14 assumes a locked configuration, whereby the second strand segment 56 applies a compressive force to the first strand segment 54. The compressive force is sufficient to prevent the first strand segment 54 from translating through the second strand segment 56 in a locked configuration. The first loop 50a is characterized as being a static loop because the size of the first loop 50a is fixed and the second loop 50b is characterized as being a dynamic loop because the size of the second loop 50b is changeable.

FIG. 35 illustrates another embodiment of a suture assembly 14a which is similar to the suture assembly 14, except that the suture assembly 14a has a first loop 50c and a second loop 50d which are both characterized as being dynamic loops. Also, the suture assembly 14a includes a first stitch lock 58a and a second stitch lock 58b, each corresponding with the first loop 50c and the second loop 50d, respectively.

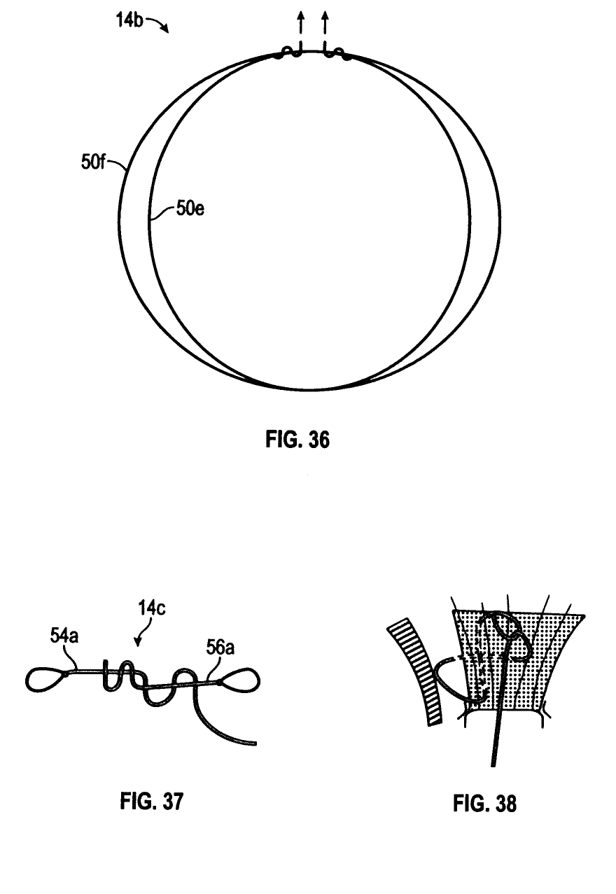

FIG. 36 illustrates another embodiment of a suture assembly 14b which is similar to the suture assembly 14a, except that the suture assembly 14b includes a first dynamic loop 50e and a second dynamic loop 50f wherein the first dynamic loop 50e and the second dynamic loop 50f are arranged in a substantially concentric relationship.

In other embodiments of the suture assembly 41c, as illustrated in FIG. 37, the strand segments 54a and 56a may be separate segments, each provided with a static loop. Also, any number of pre-tied, sliding knots commonly employed in arthroscopic surgery (e.g., a Giant knot, an SMC knot, a Weston knot) may be incorporated into the suture assembly to provide a “knotless” connection structure. For example, the use of a Mason Allen stitch is illustrated in FIG. 38. Also, a hard fastener, such as a clamp or crimp, may be employed to lock the strands.

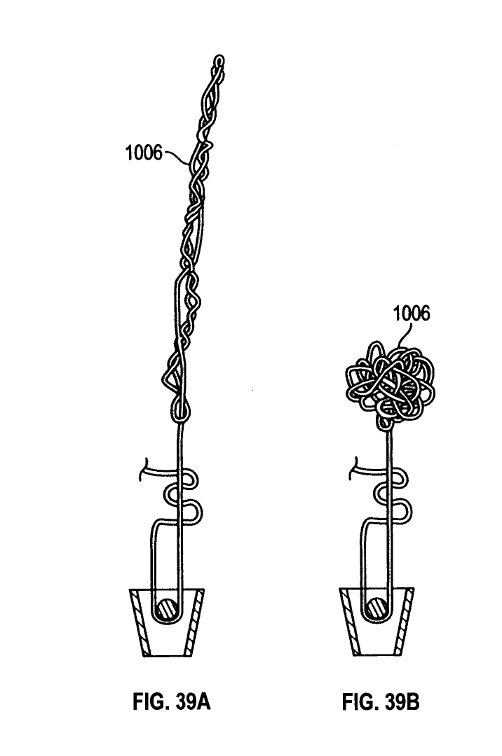

FIGS. 39A and 39B illustrate another embodiment of a suture assembly which includes an expandable portion 1006. As described in U.S. Publication No. 2012/0150223, the expandable portion 1006 may be initially placed at the target anatomical location and thereafter expanded (FIG. 39B) to secure the suture assembly to the anatomical structure, such as soft tissue or bone. By way of example, the sutures may be formed as disclosed in U.S. Publication. No. 2012/0150235, which is hereby expressly incorporated herein by reference.

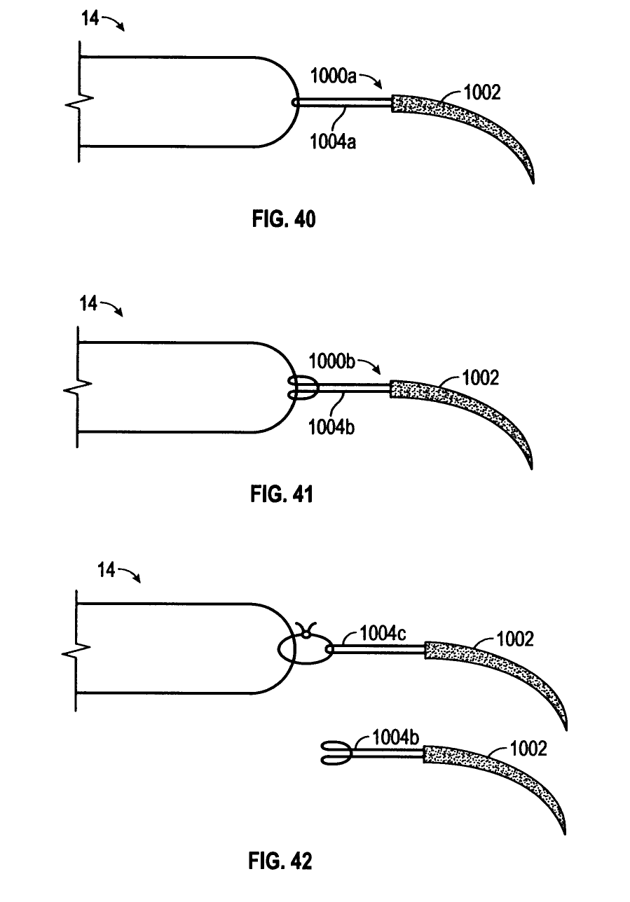

FIGS. 40-42 illustrate various embodiments of suture passers 1000a-1000c that may be used to pass the suture assembly 14 through the soft tissue 11. The suture passers 1000a-1000c may include a needle 1002 and a connector 1004. The connector 1004 may be a loop extending from the needle 1002 to connect the needle 1002 to the suture assembly 14 and thereby permit the suture assembly 14 to be passed through an anatomical structure, such as soft tissue 11. More specifically, the connector 1004 is configured to connect the needle 1002 to at least one of the loops of the suture assembly 14. As shown in FIG. 40, the connector 1004a is a loop linked to one of the loops of the suture assembly 14. The connector 1004b is threaded, tied, or looped about one of the loops of the suture assembly 14. The connector 1006c includes a second loop which may extend between the first loop of the connector 1004b and the loops of the suture assembly 14. The suture passers 1000a-1000c may be removed from the suture assembly 14 by cutting the connectors 1004. It will also be appreciated that a suture retrieving device, such as a hook-type device, may be used to pass the suture assembly 14 through the soft tissue 11.

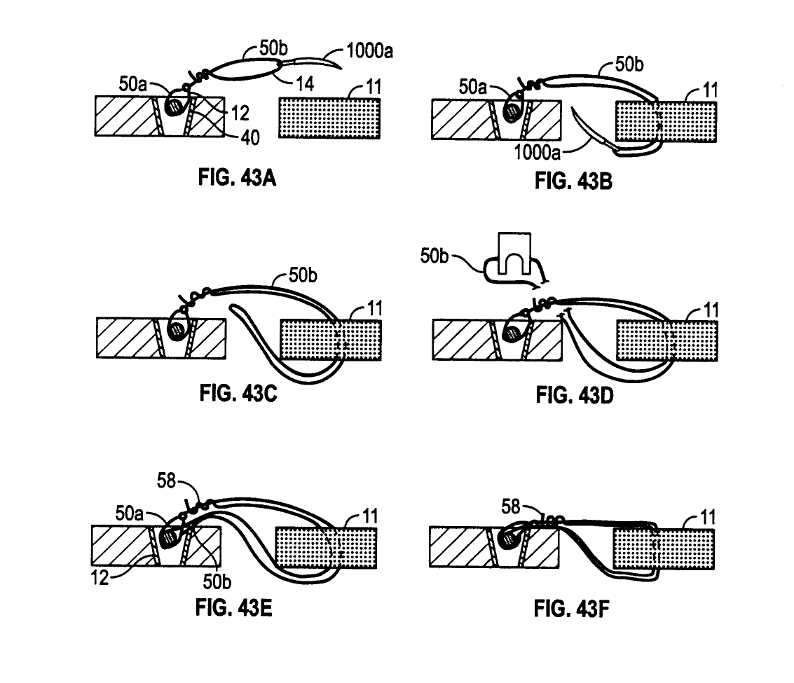

Referring now to FIGS. 43A-43F, shown therein is an exemplary method of implementing the suture anchor assembly 10 according to the inventive concepts disclosed herein. As shown in FIG. 43A, the suture anchor 12 is positioned in a bone plate. One of the loops 50a of the suture assembly 14 is captured, snared, or otherwise attached to the suture capture element 40 of the suture anchor 12. The suture assembly 14 is shown in FIG. 44A to include a suture passer 1000a as described above. The suture passer 1000a is used to pass the other loop 50b through the soft tissue 11(FIG. 43B). With the loop 50b passed through the soft tissue 11, the suture passer 1000a may be removed from the loop 50b (FIG. 43C). The loop 50b may then be deployed in the suture anchor 12, in a suitable manner such as with a suture threader, so as to capture the loop 50b about the suture capture element 40 (FIGS. 43D and 43E). With the loops 50a and 50b captured by the suture anchor 12 and the suture assembly 14 passed through the soft tissue 11, the suture assembly 14 may be tensioned as desired, such as by cinching the suture assembly 14 at the stitch lock 58 (FIG. 43F).

From the above description, it is clear that the inventive concepts disclosed and claimed herein are well adapted to carry out the objects and to attain the advantages mentioned herein, as well as those inherent in the invention. While exemplary embodiments of the inventive concepts have been described for purposes of this disclosure, it will be understood that numerous changes may be made which will readily suggest themselves to those skilled in the art and which are accomplished within the spirit of the inventive concepts disclosed and/or as defined in the appended claims.

What is claimed is:

1. An apparatus, comprising:
at least one anchor comprising a body and a suture capture element formed in the body for attaching at least one suture to the anchor, the suture capture element being configured so as to permit the suture to be snared by the capture element after the anchor has been attached to bone; and
at least one suture assembly including a first loop and a second loop, the suture assembly being configured to attach to soft tissue or bone with at least one of the first and second loops disposed about the suture capture element of the anchor.

2. The apparatus of claim 1 wherein the suture assembly comprises:
a first segment of a strand of suture; and
a second segment of a strand of suture that is elongate along a central axis so as to define a length, the first segment woven at least into the second segment along a portion of the length of the second segment so as to define a stitch lock having at least two woven segments of the first segment that are woven at least into the second segment, each of the woven segments defined at least by an entry location whereby the first segment enters the second segment, wherein the woven segments are configured to translate through the second segment strand when the second segment is in tension at a first level of tension that is less than a threshold level of tension, and the second segment applies a compressive force to the first strand when the second segment is in tension at a second level of tension that is at least substantially equal the threshold level of tension so as to prevent the first segment from translating through the second segment.

3. The apparatus of claim 2 wherein at least one of the loops is configured to decrease in size as the first segment of at least one of the first and second stitch locks is translated through the associated second segment.

4. The apparatus of claim 2, wherein the first and second segments are separate.

5. The apparatus of claim 2 wherein the first and second segments are integral with each other so as to define a common strand.

6. A method for attaching soft tissue to bone, comprising:
attaching at least one anchor to bone, the at least one anchor comprising a body and a suture capturing element attached to the body for attaching suture to the anchor, the suture-attaching element being configured so as to permit suture to be snared by the suture capturing element after the anchor has been deployed in the bone;
snaring one end of a suture assembly to the suture capturing element of the anchor;
passing the suture assembly through the soft tissue; and
snaring another end of the suture assembly to the suture capturing element of the anchor.

7. The method of claim 6, further comprising the step of cinching the suture assembly.

8. The method of claim 6, wherein body has an axial recess and a transverse edge and wherein the suture capturing element is a crossbar extending from the body and across the axial recess such that the crossbar has a free end and a fixed end and the free end of the crossbar is normally disposed distally of the transverse edge of the body, and wherein the method further comprises:
disposing the ends of the suture assembly about the crossbar in such a way that when a proximally directed force is applied to the ends of the suture assembly, the free end of the crossbar engages the transverse edge of the body to limit proximal motion of the crossbar and capture the ends of the suture assembly.

9. The method of claim 8, wherein the step of disposing the ends of the suture assembly about the crossbar further comprises the step of distally deflecting the free end of the crossbar relative to the fixed end and moving the ends of the suture assembly past the free end of the crossbar.
